(19) United States
(12) Patent Application Publication
(10) Pub. No.: US 2024/0189487 A1
(43) Pub. Date: Jun. 13, 2024

(54) STOMA SKIN SAVER COMPOSITION

(71) Applicant: Jasmine's Lab Incorporated, Whittier, CA (US)

(72) Inventors: Jasmine Rowan Sturr, Whittier, CA (US); Melvin Rowan Sturr, Whittier, CA (US)

(73) Assignee: Jasmine's Lab Incorporated, Whittier, CA (US)

(21) Appl. No.: 18/222,692

(22) Filed: Jul. 17, 2023

Related U.S. Application Data
(60) Provisional application No. 63/430,697, filed on Dec. 7, 2022.

(51) Int. Cl. A61L 28/00

(52) U.S. Cl. CPC A61L 28/0003; A61L 28/0019; A61L 28/0034

(57) ABSTRACT

A method includes identifying a stoma at an initial position. The method also includes identifying one or more portions of a leak originating from the stoma at an initial position. The method further includes applying a composition to the one or more portions of the leak. The composition includes bentonite clay and guaran. The bentonite clay and guaran enable the composition to soak the one or more portions of the leak, and prevent the one or more portions from continuing to flow. The method also includes determining whether additional portions of the leak have originated from the stoma after the composition has been applied.

### INCORPORATION BY REFERENCE

This application claims priority to the provisional application, entitled, “Stoma Skin Saver Composition,” and application number 63/430,697, filed on Dec. 7, 2022.

### TECHNICAL FIELD

The present disclosure generally relates to preventing leaks from occurring around a stoma region of one or more patients.

### BACKGROUND

Leaks can occur in various regions of patients. The leaks can occur and continue to occur in various areas of the patient. When leakage is in contact with skin membrane, it begins to cause irritation and skin breakdown, which are painful to the patient and can lead to treatment complications. After an interval when the leak has been temporarily stopped, the portions of the leak can continue to occur in various regions of the patient.

Patients can face the problems of having to continuously have to stop the leak in multiple time intervals. Each attempt to stop the leak could be a temporary solution. As such, patients can be faced with the challenge of having to always stop the leaks for a temporary period. Current solutions are inadequate to stop leakages while preventing the fluids from pooling on the skin. Formulations that exist to repair the skin and aid in wound repair do not address the root cause of preventing the leaking fluids from continued contact with the skin.

The solutions that exist for treating leakage around colostomy bags specifically do not work on other types of stomas and weeping wounds, causing additional pain for some patients if attempted to use on other areas The patients can face side effects that can adversely affect portions of their skin and other parts of their body.

As such, a need exits an effective solution that minimizes the number of times that patients have to use to stop leaks from occurring. In addition, a need exists for a solution that will not have any harmful side effects for patients as well.

### SUMMARY

The following summary is provided to facilitate an understanding of some of the features of the disclosed embodiments and is not intended to be a full description. A full appreciation of the various aspects of the embodiments disclosed herein can be gained by taking the specification, claims, drawings, and abstract as a whole.

The aforementioned aspects and other objectives can now be achieved as described herein.

In an embodiment, a method includes identifying a stoma at an initial position. The method also includes identifying one or more portions of a leak that are originating from the stoma at the initial position. The method also includes applying a composition to the one or more portions of the leak originating from the stoma. The composition includes bentonite clay and guaran. The bentonite clay and guaran enable the composition to soak the one or more portions of the leak and prevent the one or more portions from continuing to flow. The method also includes determining whether additional portions of the leak have originated from the stoma after the composition has been applied.

In another embodiment, a method includes applying at least one of a bentonite clay, guaran, and/or arrowroot powder into a single composition. The method also includes identifying one or more positions around a stoma to apply the composition. The one or more positions of a leak are identified at the one or more positions around the stoma to apply the composition. The method also includes applying the composition to the one or more positions of the leak around the stoma. Further, the method includes identifying one or more time intervals at which the composition absorbs the one or more portions of the leak around the stoma.

In yet another embodiment, a system includes a stoma at an initial position on one or more skin surfaces. The system also includes one or more portions of a leak originating from the stoma at an initial position. Further, the system includes a composition applied to the one or more portions of the leak that are originating from the stoma. The composition includes bentonite clay and guaran. The bentonite clay and guaran enable the composition to soak the one or more portions of the leak, and prevent the one or more portions from continuing to flow. The system also includes additional portions of the leak that have originated from the stoma after the composition has been applied.

### BRIEF DESCRIPTION OF THE FIGURES

The accompanying figures, in which like reference numerals refer to identical or functionally similar elements throughout the separate views and which are incorporated and form a part of the specification, further illustrate the present invention and, together with the detailed description of the invention, serve to explain the principles of the present invention.

FIG. 1 illustrates a schematic diagram in accordance with an embodiment of the invention;

FIG. 2 illustrates another schematic diagram in accordance with an embodiment of the invention;

FIG. 3 illustrates a flowchart in accordance with an embodiment of the invention;

FIG. 4 illustrates a flow chart in accordance with an embodiment of the invention;

FIG. 5 illustrates another flow chart in accordance with an embodiment of the invention;

FIG. 6 illustrates a flow chart in accordance with an embodiment of the invention;

FIG. 7 depicts another flow chart in accordance with an embodiment of the invention;

FIG. 8 illustrates a flow chart in accordance with an embodiment of the invention;

FIG. 9 depicts a diagram in accordance with an embodiment of the invention;

FIG. 10 illustrates a diagram in accordance with an embodiment of the invention;

FIG. 11 depicts another diagram in accordance with an embodiment of the invention;

FIG. 12 depicts a flow chart in accordance with an embodiment of the invention;

FIG. 13 illustrates another flow chart in accordance with an embodiment of the invention;

FIG. 14 depicts a diagram in accordance with an embodiment of the invention;

FIG. 15 illustrates another diagram in accordance with an embodiment of the invention; and

FIG. 16 illustrates a diagram in accordance with an embodiment of the invention.

Unless otherwise indicated illustrations in the figures are not necessarily drawn to scale.

### DETAILED DESCRIPTION

The particular values and configurations discussed in these non-limiting examples can be varied and are cited merely to illustrate one or more embodiments and are not intended to limit the scope thereof.

Subject matter will now be described more fully herein after with reference to the accompanying drawings, which form a part hereof, and which show, by way of illustration, specific example embodiments. Subject matter may, however, be embodied in a variety of different form and, therefore, covered or claimed subject matter is intended to be construed as not being limited to any example embodiments set forth herein, example embodiments are provided merely to be illustrative. Likewise, a reasonably broad scope for claimed or covered subject matter is intended. Among other issues, subject matter may be embodied as methods, devices, components, or systems. The followed detailed description is, therefore, not intended to be interpreted in a limiting sense.

Throughout the specification and claims, terms may have nuanced meanings suggested or implied in context beyond an explicitly stated meaning. Likewise, phrases such as “in one embodiment” or “in an example embodiment” and variations thereof as utilized herein may not necessarily refer to the same embodiment and the phrase “in another embodiment” or “in another example embodiment” and variations thereof as utilized herein may or may not necessarily refer to a different embodiment. It is intended, for example, that claimed subject matter include combinations of example embodiments in whole or in part.

In general, terminology may be understood, at least in part, from usage in context. For example, terms such as “and,” “or,” or “and/or” as used herein may include a variety of meanings that may depend, at least in part, upon the context in which such terms are used. Generally, “or” if used to associate a list, such as A, B, or C, is intended to mean A, B, and C, here used in the inclusive sense, as well as A, B, or C, here used in the exclusive sense. In addition, the term “one or more” as used herein, depending at least in part upon context, may be used to describe any feature, structure, or characteristic in a singular sense or may be used to describe combinations of features, structures, or characteristics in a plural sense. Similarly, terms such as a “a,” “an,” or “the”, again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

One having ordinary skill in the relevant art will readily recognize the subject matter disclosed herein can be practiced without one or more of the specific details or with other methods. In other instances, well-known structures or operations are not shown in detail to avoid obscuring certain aspects. This disclosure is not limited by the illustrated ordering of acts or events, as some acts may occur in different orders and/or concurrently with other acts or events. Furthermore, not all illustrated acts or events are required to implement a methodology in accordance with the embodiments disclosed herein.

Unless defined otherwise, all technical and scientific terms used herein have the same meanings as commonly understood by one of ordinary skill in the art to which the disclosed embodiments belong. Preferred methods, techniques, devices, and materials are described, although any methods, techniques, devices, or materials similar or equivalent to those described herein may be used in the practice or testing of the present invention.

Although claims have been included in this application to specific enumerated combinations of features, it should be understood the scope of the present disclosure also includes any novel feature or any novel combination of features disclosed herein.

References “an embodiment,” “example embodiment,” “various embodiments,” “some embodiments,” etc., may indicate that the embodiment(s) so described may include a particular feature, structure, or characteristic, but not every possible embodiment necessarily includes that particular feature, structure, or characteristic.

Headings provided are for convenience and are not to be taken as limiting the present disclosure in any way.

Each term utilized herein is to be given its broadest interpretation given the context in which that term is utilized.

### Terminology

The following paragraphs provide context for terms found in the present disclosure (including the claims):

The transitional term “comprising”, which is synonymous with “including,” “containing,” or “characterized by,” is inclusive or open-ended and does not exclude additional, unrecited elements or method steps. See, e.g., Mars Inc. v. H.J. Heinz Co., 377 F.3d 1369, 1376, 71 USPQ2d 1837, 1843 (Fed. Cir. 2004) (“[L]ike the term ‘comprising,’ the terms ‘containing’ and ‘mixture’ are open-ended.”). “Configured to” or “operable for” is used to connote structure by indicating that the mechanisms/units/components include structure that performs the task or tasks during operation. “Configured to” may include adapting a manufacturing process to fabricate components that are adapted to implement or perform one or more tasks.

“Based On.” As used herein, this term is used to describe factors that affect a determination without otherwise precluding other or additional factors that may affect that determination. More particularly, such a determination may be solely “based on” those factors or based, at least in part, on those factors.

All terms of example language (e.g., including, without limitation, “such as”, “like”, “for example”, “for instance”, “similar to”, etc.) are not exclusive of other examples and therefore mean “by way of example, and not limitation . . . ”

A description of an embodiment having components in communication with each other does not infer that all enumerated components are needed.

A commercial implementation in accordance with the scope and spirit of the present disclosure may be configured according to the needs of the particular application, whereby any function of the teachings related to any described embodiment of the present invention may be suitably changed by those skilled in the art.

The flowchart and block diagrams in the figures illustrate the architecture, functionality, and operation of possible implementations of systems and methods according to various embodiments. Functions noted in the block may occur out of the order noted in the figures. For example, two blocks shown in succession may, in fact, be executed substantially concurrently, or the blocks may sometimes be executed in the reverse order, depending upon the functionality involved.

Further, any sequence of steps that may be described does not necessarily indicate a condition that the steps be performed in that order. Some steps may be performed simultaneously.

The functionality and/or the features of a particular component may be alternatively embodied by one or more other devices that are not explicitly described as having such functionality/features. Also, various embodiments of the present invention need not include a device itself.

More specifically, as will be appreciated by one skilled in the art, aspects of the present invention may be embodied as a system and/or method. Furthermore, aspects of the present invention may take the form of a plurality of systems.

### INTRODUCTION

Embodiments of include a composition put together using a plurality of components. The plurality of components include bentonite clay, arrow root powder, guaran, and sodium alginate. The composition has a set percentage for each of the components. As such, bentonite clay comprises sixty percent, guaran twenty percent, sodium alginate ten percent, and arrowroot powder comprises ten percent. The bentonite clay is a swelling agent, adsorbing leakage and exudate into the folds of the material upon exposure. The guaran and sodium alginate are natural polymers, with the guaran being a polysaccharide with multiple functional sugar groups. Upon activation by liquid exudate, it forms a complex three dimensional gel matrix. The sodium alginate is a marine derived polymer that upon activation with liquid exudate reacts to transform from a low viscosity solution to a complex gel structure, particularly known for its emulsifying and hydrocolloid properties. The arrowroot retains properties to à lesser degree than the previous components and provides odor control in addition.

The composition is blended together and the placed in a dispensing device. The dispensing device disposes the composition onto or around a stoma region of patients. The stoma region has a leak or portions of the leak that are leaking around the stoma region. The composition is displaced onto the stoma and/or around the stoma region to stop and/or absorb portions of the leak.

The applied composition absorbs portions of the leak in and around the stoma. In addition, the applied composition stops additional portions of the leak from occurring around the stoma. If other portions of the leak continue to occur, additional applications of the composition onto and/or around the stoma region occur.

A gauze like device is placed on the composition after the composition absorbs and prevents the leaks. The user determines in one or more intervals whether additional applications of the composition should be applied.

### System Description

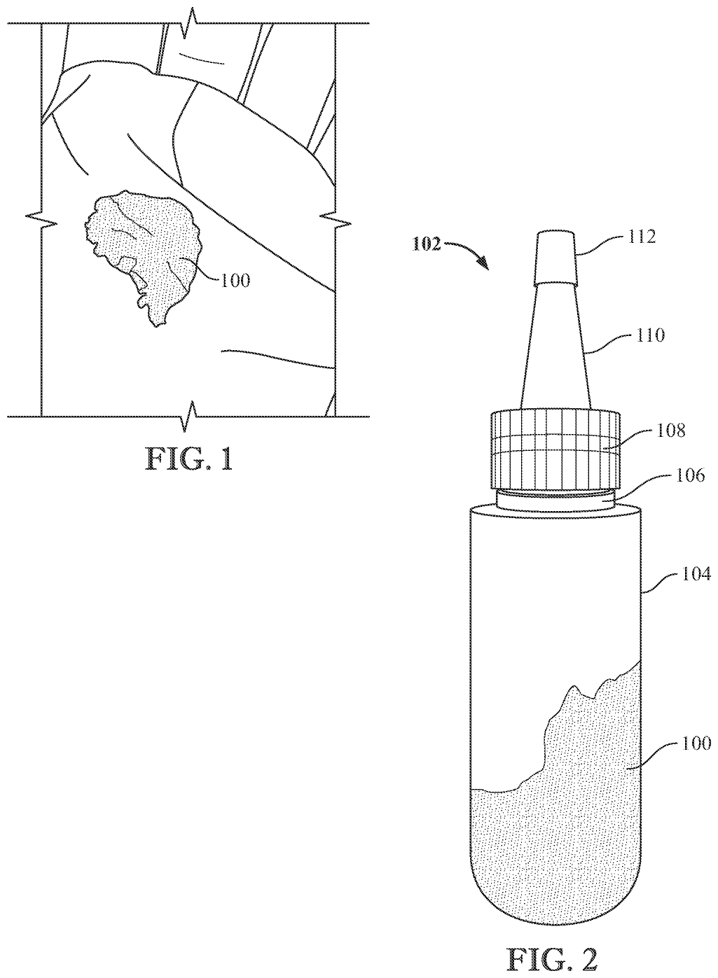

FIG. 1 illustrates a stoma with the composition 100 applied to the stoma. The leak of the stoma is identified by the user. The user also identifies how many portions of the leak are occurring from the stoma. The leak can be a single leak in one direction. In other embodiments, the leak can have multiple portions that are extending in a plurality of directions. The user identifies the full exterior region of the leak. In particular, the user identifies the how far that the leak extends from the stoma.

Referring to FIG. 1, the user positions the composition 100 over the entire portion of the leak that originates from the stoma. The user places bentonite clay, arrow root power, guaran, and sodium alginate within the composition. The bentonite clay has the greatest ratio among the components of the composition 100. As such, the user applies the composition 100 to soak and absorb the entire portion of the leak. After the composition 100 is applied, the user determines if any additional portions of the leak have occurred. If the additional portions of the leak have occurred, the user applies additional portions of the composition 100. Alternatively, if the leak has stopped, the user determines that no additional portions of the composition 100 are required.

Referring to FIG. 2, the container 102 which holds the composition 100 is illustrated. Within the container, 102, the composition 100 is shown at the bottom of the container 102. The container 102 also includes a tube 104 that holds the composition 100. The container 102 also includes an inner cover 106 over the tube 104. Further, the container 102 includes an outer portion 108 and dispenser 110. The composition 100 will flow through the dispenser 110 when the user is disposing the composition 100 onto or around the stoma region. In addition, the user also dispenses the composition 100 through the tip 112 of the container 102.

In FIG. 2, the composition 100 within the container 102 will be dispensed through the dispenser 110 and the tip 112 of the container 102. The composition 100 is applied on the entire portion or around the entire portion of the leak that occurs on and around the stoma. Further, the composition 100 will also be re-applied when additional portions of the leak occur or resurface on the stoma region.

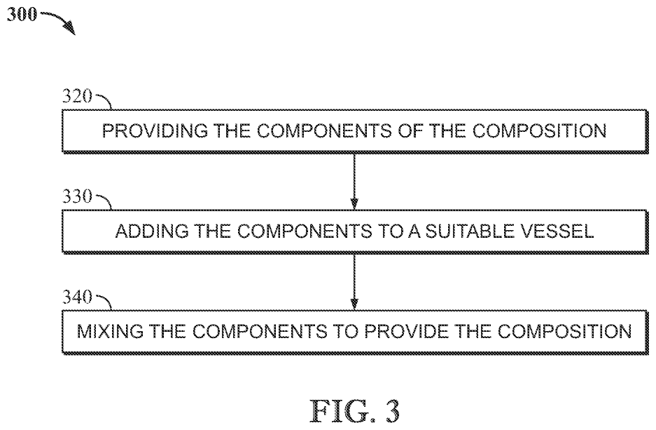

Referring to FIG. 3, a flowchart 300 is illustrated that describes a process of the composition. At step 320, the components of the composition are provided. The components include bentonite clay, guaran, arrowroot powder, and sodium alginate. The bentonite clay comprises sixty percent of the composition. Further, guaran comprises twenty percent of the composition. The arrowroot powder and sodium alginate each comprise ten percent of the composition.

Still referring to FIG. 3, at step 330, the components are provided to a suitable vessel. The vessel includes a bowl or tube of suitable dimensions. Further, at step 340, the components are mixed together to provide the composition. As mentioned above, the bentonite clay comprises sixty percent of the composition.

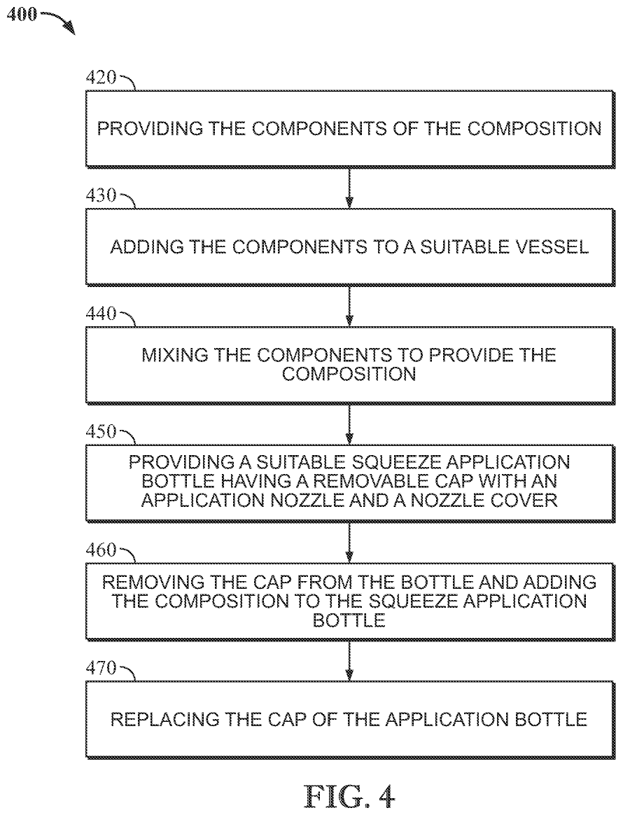

In FIG. 4, another flowchart 400 is illustrated. The flowchart 400 illustrates how the composition is put together, and then placed in a proper dispensing instrument to apply to a stoma region of one or more individuals. At step 420, the components of the composition are provided. The components include bentonite clay, guaran, sodium alginate, and arrowroot powder. The bentonite clay comprise sixty percent, guaran twenty percent, and sodium alginate twenty percent of the composition. At step 430, the components are added to a suitable vessel such as tube dispensing device.

Still referring to FIG. 4, at step 440, the components are mixed to provide the composition. The bentonite clay, guaran, arrowroot powder, and sodium alginate are mixed altogether to form the composition. The bentonite clay comprises sixty percent of the composition. At step 450, a suitable squeeze application bottle is provided for the composition. The bottle includes a removable cap. In addition, the bottle also includes an application nozzle and a nozzle cover to ensure that it is easy to dispense the composition onto the stoma region. At step 460, the cap from the bottle is removed, and the composition is added to the bottle. The composition is positioned in the bottle to eventually be disposed on the stoma region. Further, at step 470, the cap of the application bottle is replaced.

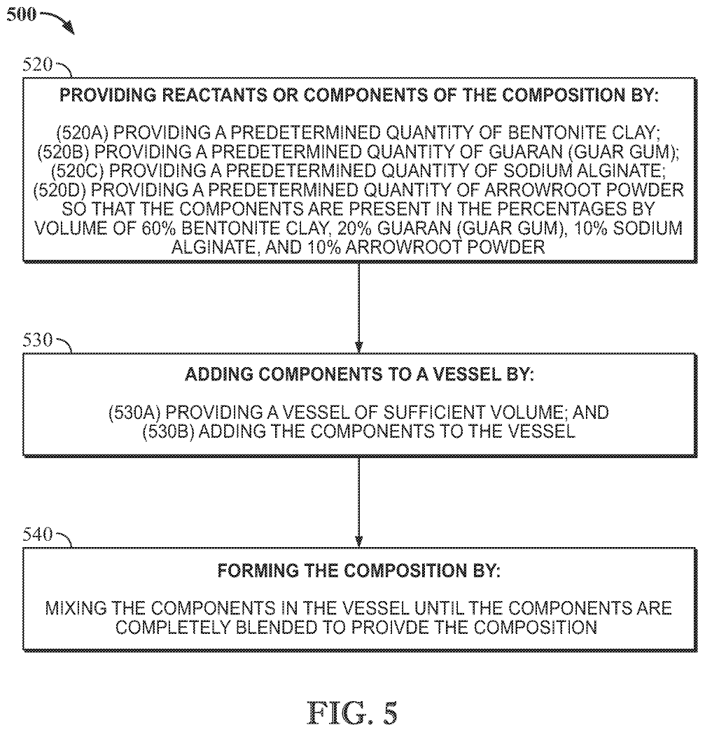

In FIG. 5, another process 500 is illustrated with respect to the composition. At step 520, the predetermine quantity of the bentonite clay, guaran, arrowroot powder, and sodium alginate is illustrated. As previously mentioned, bentonite clay comprises sixty percent, guaran twenty percent, sodium alginate ten percent, and arrowroot powder ten percent. Further, at step 530, the components are added to the vessel or dispensing tube. A vessel 530A of sufficient volume is provided, and the components 530B are added to the vessel. At step 540, the composition is formed by mixing all of the components together until they are completely blended to form the composition.

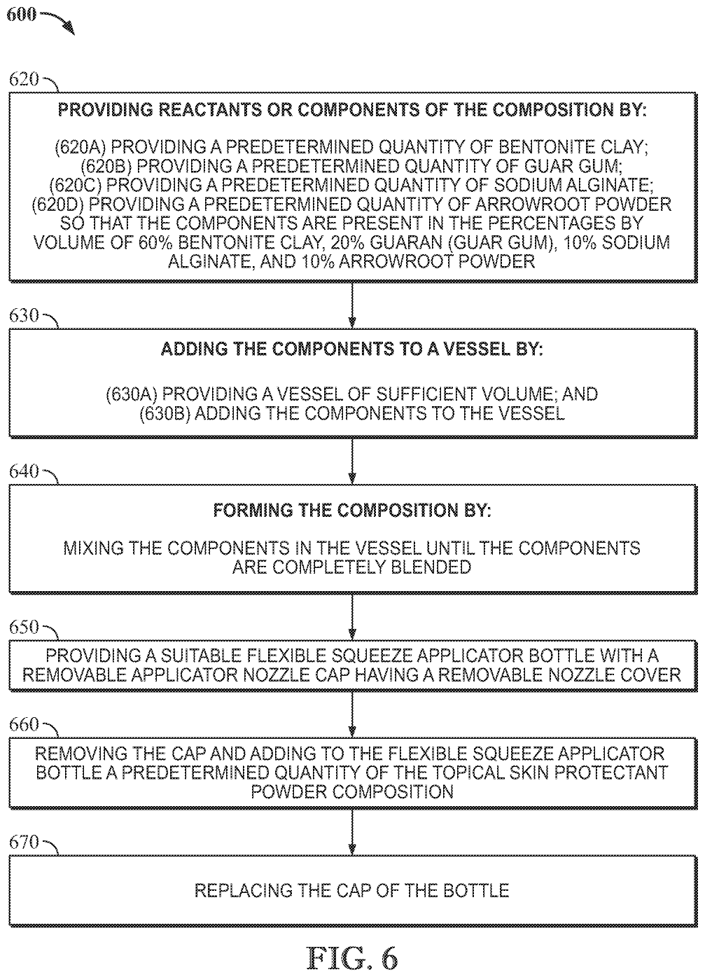

Referring to FIG. 6, another process 600 is illustrated. The process 600 illustrates how the composition is provided to a squeeze applicator bottle. At step 620, the predetermined quantity of bentonite clay 620A, Guaran 620B, predetermined quantity sodium alginate 620C, and predetermined quantity of arrowroot powder 620D is shown. At step 630, the components are added to the vessel with a vessel 630A of sufficient volume, and the components 630B being added to the vessel. At step 640, the composition is formed by mixing the components together. Next, at step 650, a suitable squeeze applicator bottle is provided. At step 660, a predetermined quantity of the topical skin protectant powder composition is provided to the applicator bottle. Further, at step 670, the cap of the bottle is replaced after the composition is added.

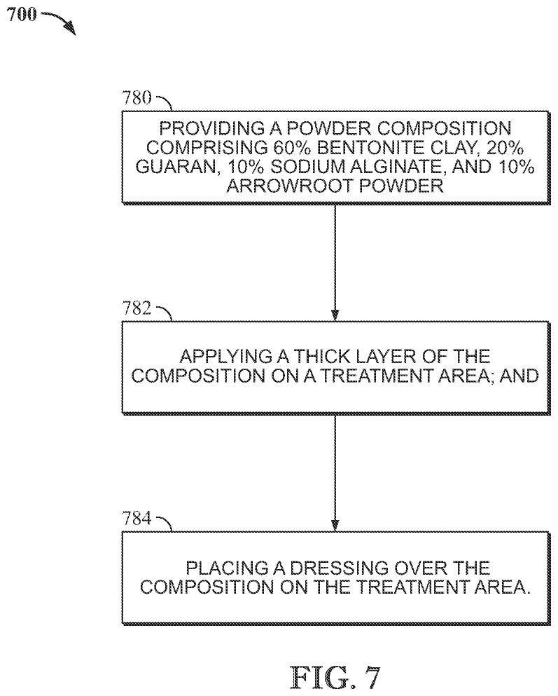

In FIG. 7, yet another process 700 is illustrated. The process 700 illustrates how the composition is added to treat a stoma area of a patient. At step 780, a powder composition is provided. The composition includes bentonite clay at sixty percent, guaran at twenty percent, sodium alginate at ten percent, and arrowroot powder at ten percent. Next, at step 782, a thick layer of the composition is applied on or around the treatment area. The treatment area is on the stoma of the patient. At step 784, a dressing over the composition is placed on the treatment area by the patient or caretaker. The process 700 illustrates placing the composition on the stoma region to treat the patient, and then applying a dressing on the stoma area after the composition has been provided.

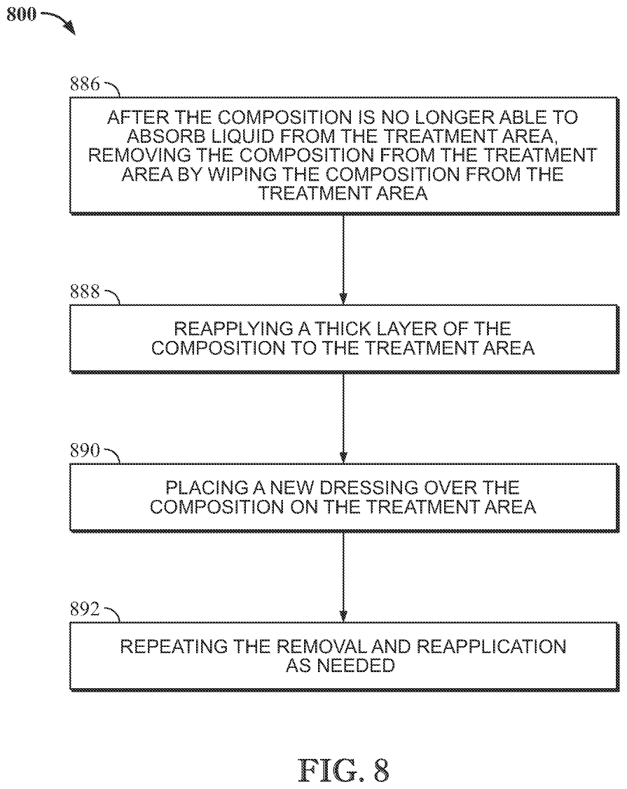

Referring to FIG. 8, another process 800 is illustrated. The process 800 illustrates what occurs after the composition has been applied to the stoma region of the patient. At step 886, after the composition is no longer able to absorb liquid from the treatment area, the composition is removed from the treatment area by wiping the composition from the stoma or treatment area. Then, at step 888, the composition is reapplied to the treatment area. A thick layer of the composition is reapplied in or around the stoma region. At step 890, a new dressing is placed over the composition area. The leak portions have been absorbed by the composition. At step 892, the removal and reapplication of the composition is applied as needed. If the leak portions have stopped or been absorbed, the reapplication can stop. Alternatively, the composition process 800 is repeated if the leak portions have continued.

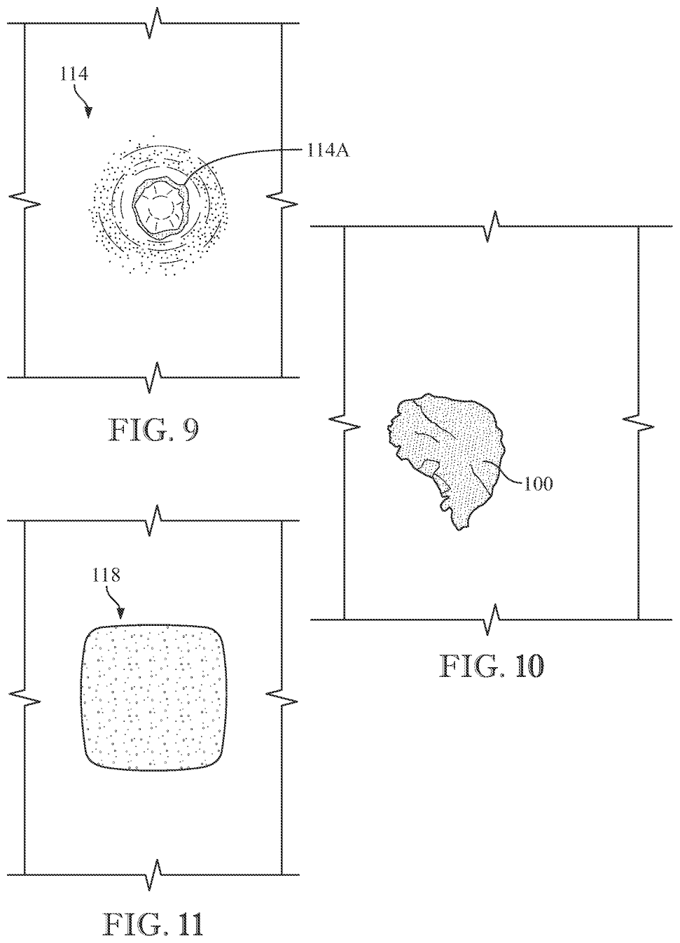

Referring to FIG. 9, a front view of the procedure is shown. Moreover, the composition 114A is shown to be applied to or around the stoma region of the patient to absorb and discontinue the leak that is occurring or has occurred. The dressing 114 is also shown. The procedure is repeated as many times as necessary until the leak has been stopped and absorbed.

In FIG. 10, the composition 100 is positioned over the stoma area. In this illustration, the composition 100 has absorbed all portions of the leak from the stoma, and no portion of the leak remain. The composition 100 is reapplied as necessary until the leak portion or portions have stopped.

Referring to FIG. 11, another illustration of the composition 118 is shown. A different shape of the composition 118 is shown as it is positioned over the stoma. The user determines the shape of distribution of the composition 118 around the stoma. The user determines what shape is required to enable the composition 118 to effectively absorb and stop the leak portion or portions.

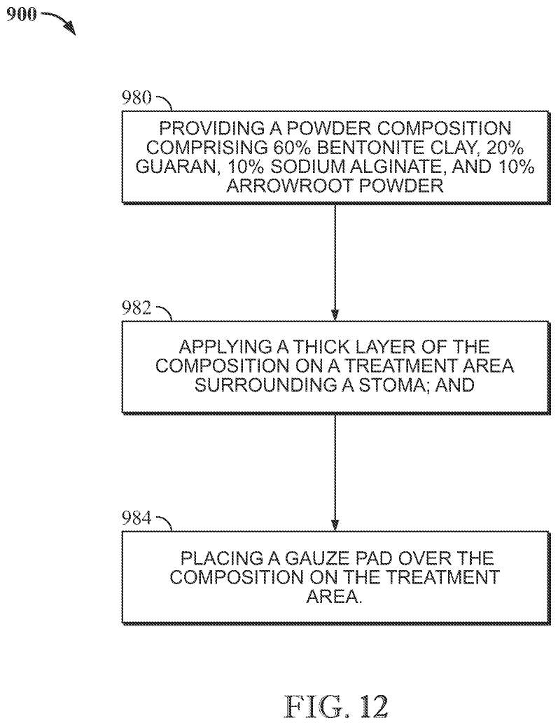

In FIG. 12, another process 900 is shown. At step 980, the composition is provided with sixty percent bentonite clay, twenty percent guaran, ten percent sodium alginate, and ten percent arrowroot powder. After the composition is placed in a dispensing apparatus, at step 982, a thick layer of the composition is applied on a treatment area on or around the stoma to absorb all portions of a leak. Further, at step 984, the user places a gauze pad over the composition on the treatment area after the leak portions have been absorbed.

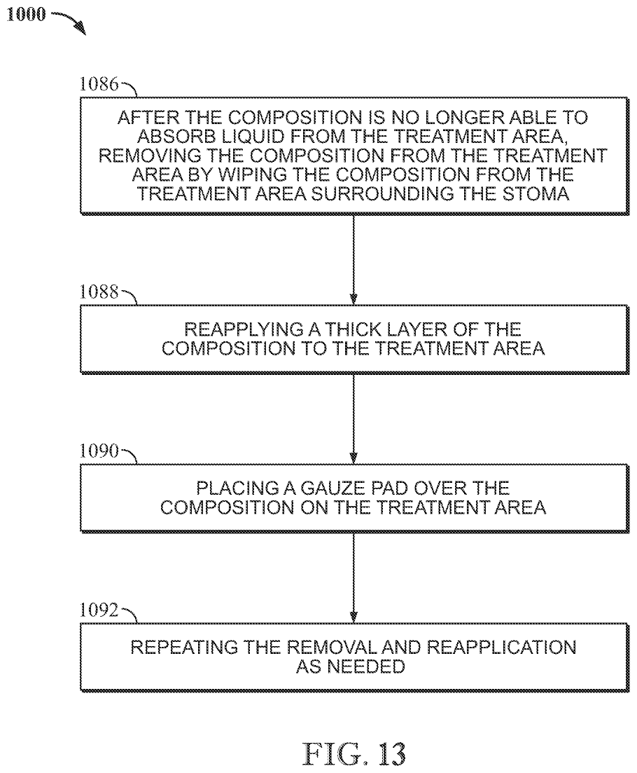

Referring to FIG. 13, another process 1000 is illustrated. At step 1086, after the composition is no longer able to absorb the liquid from the leak, the composition from the treatment area surrounding the stoma is removed. The user wipes the composition from the treatment area surrounding the stoma. At step 1088, the composition is reapplied to the treatment area to prevent further portions of the leak. Further, at step 1090, a gauze pad is placed over the composition on the treatment areas. At step 1092, the process is repeated as necessary with the removal of the composition and reapplication of the composition on the treatment area until all portions of the leak have been absorbed or stopped.

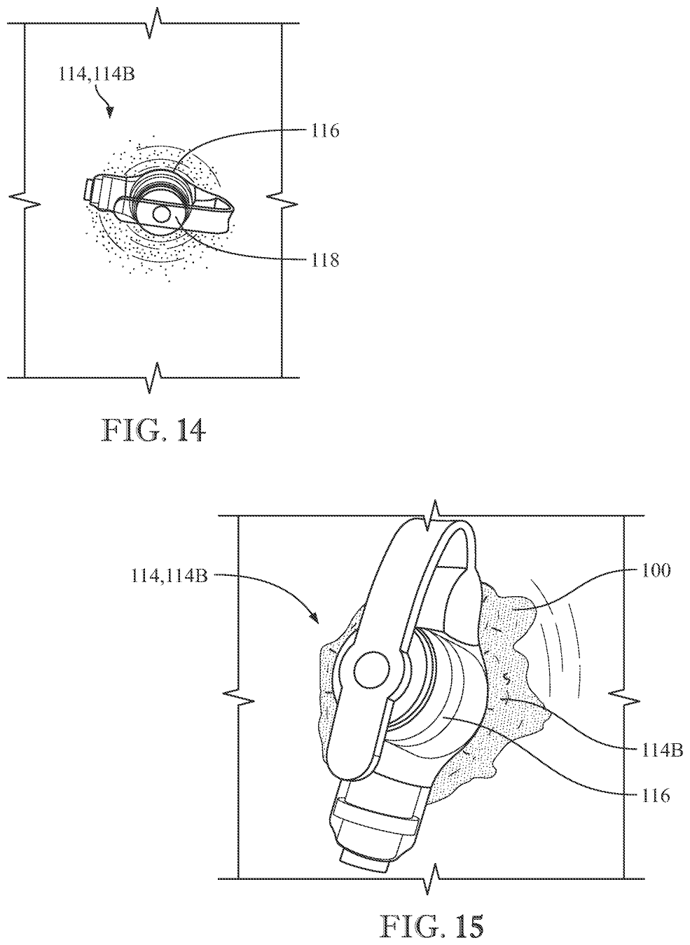

In FIG. 14, another illustration is shown. Moreover, skin 114, 114B affecting by the seepage of the leak is illustrated. In addition, a gauze type device 116 is shown positioned over the skin 114, 114B affected by the seepage. In addition, the skin 118 affected by the leakage from the stoma such as a bedsore or draining wound that is causing a skin breakdown is also shown.

Referring to FIG. 15, another illustration of the composition 100 is shown. The skin 114, 114B affected by the seepage of the leak around the stoma is shown. In addition, the gauze type device 116 over the skin 114, 114B and composition 100 is also shown. The gauze type device 116 is placed after one or more portions of the leak have been stopped.

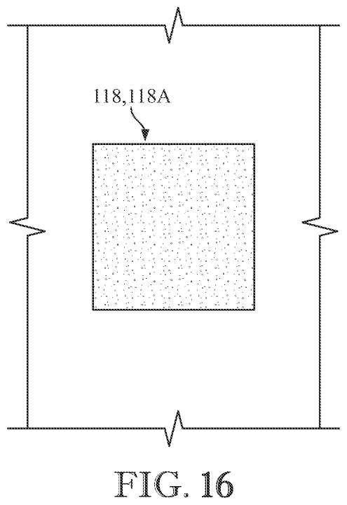

In FIG. 16, another illustration is shown. Moreover, skin 118, 118A is shown that is affected by leakage such as a bedsore or a draining wound that is causing a skin breakdown.

Those skilled in the art will appreciate that the example embodiments are non-exhaustive and that embodiments other than that described here may be included without departing from the scope and spirit of the presently disclosed embodiments.

### Advantages/Summary

A process is illustrated of identifying a leak or leak portions in a stoma region of a patient. The stoma region can be anywhere on the patient. The leak can be in a variety of directions and have multiple portions originating from the stoma region. After the leak is identified, the user treats the stoma region with the composition.

The user identifies the components and proportion of the components to place within the composition. The components includes bentonite clay, guaran, sodium alginate, and arrow root powder. Moreover, the bentonite clay comprises sixty percent, guaran twenty percent, sodium alginate ten percent, and arrowroot powder ten percent. The bentonite clay is the primary component in the composition that enables the composition to absorb and stop the leak portions or portions from occurring on the stoma region. As mentioned above, the bentonite clay is a swelling agent, adsorbing leakage and exudate into the folds of the material upon exposure. Further, the guaran and sodium alginate are natural polymers, with the guaran being a polysaccharide with multiple functional sugar groups. Moreover, upon activation by liquid exudate, it forms a complex three dimensional gel matrix. The sodium alginate is a marine derived polymer that upon activation with liquid exudate reacts to transform from a low viscosity solution to a complex gel structure, particularly known for its emulsifying and hydrocolloid properties. The arrowroot retains properties to a lesser degree than the previous components and provides odor control in addition.

Once the composition is put together, and blended together, the composition is placed in a dispensing device. A variety of dispensing devices can be used. The user determines the best dispensing device to dispose the composition onto the stoma.

Overall, the composition is used to stop the one or more portions of the leak in and/or around the stoma. A gauze like device is placed on the stoma region after the composition has stopped the one or more portions of the leak.

Benefits to the patient include odor suppression. In addition to preventing skin breakdown, preventing leakage also prevents clothing and bedding from staining.

### CONCLUSION

All references, including granted patents and patent application publications, referred herein are incorporated herein by reference in their entirety.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

Various aspects of the invention have been described above by way of illustration, and the specific embodiments disclosed are not intended to limit the invention to the particular forms disclosed. The particular implementation of the system provided thereof may vary depending upon the particular context or application. The invention is thus to cover all modifications, equivalents, and alternatives falling within the spirit and scope of the following claims. It is to be further understood that not all of the disclosed embodiments in the foregoing specification will necessarily satisfy or achieve each of the objects, advantages, or improvements described in the foregoing specification.

The corresponding structures, materials, acts, and equivalents of all means or step plus function elements in the claims below are intended to include any structure, material, or act for performing the function in combination with other claimed elements as specifically claimed.

1. A method comprising:
identifying a stoma at an initial position;
identifying one or more portions of a leak that are originating from the stoma at the initial position:
applying a composition to the one or more portions of the leak that are originating from the stoma, wherein the composition includes bentonite clay and guaran, wherein the bentonite clay and guaran enable the composition to soak the one or more portions of the leak, and prevent the one or more portions from continuing to flow; and
determining whether additional portions of the leak have originated from the stoma after the composition has been applied.

2. The method of claim 1, further comprising:
applying a predetermined amount of arrowroot powder to the composition before it is applied to the one or more portions of the leak.

3. The method of claim 1, wherein the composition includes a greater amount of bentonite clay than guaran.

4. The method of claim 1, further comprising:
mixing the bentonite clay and guaran into a blended composition before applying the composition to the one or more portions of the leak.

5. The method of claim 1, further comprising:
applying the composition to the one or more portions of the leak in various time intervals.

6. The method of claim 1, further comprising:
identifying additional portions of the leak after applying the composition.

7. The method of claim 1, furthering comprising:
identifying a time interval when the composition has absorbed the one or more portions of the leak.

8. A method comprising:
applying at least one of a bentonite clay, guaran, and/or arrowroot power into a single composition;
identifying one or more positions around a stoma to apply the composition, wherein one or more portions of a leak are identified at the one or more positions around the stoma to apply the composition;
applying the composition to the one or more portions of the leak around the stoma; and
identifying one or more time intervals at which the composition absorbs the one or more portions of the leak around the stoma.

9. The method of claim 8, further comprising:
assigning a ratio of the bentonite clay in relation to the arrowroot powder before applying the bentonite clay and arrowroot powder into the composition.

10. The method of claim 8, further comprising:
identifying an end point for each of the one or more leaks around the stoma.

11. The method of claim 8, wherein the stoma is a position on a region of one or more patients.

12. The method of claim 8, further comprising:
providing a greater amount of bentonite clay than guaran to the composition.

13. The method of claim 8, further comprising:
identifying one or more additional portions of the leak after applying the composition.

14. The method of claim 8, further comprising:
securing each portion of the composition around the stoma to prevent further leaking from the one or more portions of the leak.

15. A system comprising:
a stoma at an initial position on one or more skin surfaces;
one or more portions of a leak that are originating from the stoma at the initial position:
a composition applied to the one or more portions of the leak that are originating from the stoma, wherein the composition includes bentonite clay and guaran, wherein the bentonite clay and guaran enable the composition to soak the one or more portions of the leak, and prevent the one or more portions from continuing to flow; and
additional portions of the leak that have originated from the stoma after the composition has been applied.

16. The system of claim 15, wherein additional portions of the composition are applied to the additional portions of the leak.

17. The system of claim 15, wherein sodium alginate is also provided within the composition.

18. The system of claim 15, wherein a higher level of bentonite clay is provided within the composition than guaran.

19. The system of claim 15, wherein a gauze pad is placed over the composition.

20. The system of claim 15, wherein the composition is redistributed over the one or more portions of the leak.
